(19) United States
(12) Patent Application Publication
(10) Pub. No.: US 2024/0047040 A1
(43) Pub. Date: Feb. 8, 2024

(54) INFORMATION PROCESSING SYSTEM AND INFORMATION PROCESSING METHOD

(71) Applicant: Hitachi, Ltd., Tokyo (JP)

(72) Inventors: Zisheng LI, Tokyo (JP); Masahiro OGINO, Tokyo (JP)

(21) Appl. No.: 18/268,669

(22) PCT Filed: Nov. 2, 2021
(86) PCT No.: PCT/JP2021/040378

§ 371 Date: Jun. 21, 2023

(30) Foreign Application Priority Data
Feb. 10, 2021 (JP) 2021-019921

(51) Int. Cl. G16H 20/70; G06N 5/022

(52) U.S. Cl. CPC G16H 20/70; G06N 5/022

(57) ABSTRACT

An information processing system includes: a behavior history data feature extraction unit configured to extract a behavior history data feature that is a feature of the behavior history data acquired from the user; a measurement data feature extraction unit configured to extract a measurement data feature that is a feature of the measurement data acquired from the user; a feature conversion learning unit configured to learn a feature conversion model for deriving a feature of measurement data from the behavior history data using the behavior history data feature and the measurement data feature; and a treatment prediction learning unit configured to generate a prediction model for providing an appropriate treatment to a user using a first feature extracted from the measurement data, a second feature converted from the behavior history data, the treatment, and an effect of the treatment.

### INCORPORATION BY REFERENCE

The present application claims priority based on Japanese Patent Application No. 2021-19921 filed Feb. 10, 2021, the content of which is incorporated herein by reference.

### TECHNICAL FIELD

The present invention relates to an information processing system and particularly relates to an information processing system that proposes an appropriate treatment not only using measurement data of a user but also using behavior history data.

### BACKGROUND ART

Recently, as the productive age population decreases, labor shortage becomes serious, and the productivity of individual employees is required to be improved for companies. However, actually, the state of mind and body deteriorates depending on living conditions, working conditions, or the like, and thus the productivity may decrease.

To prevent the decrease in productivity, a treatment of adjusting effect factors such as living conditions or working conditions is required. However, the effect factors or the states of mind and body of individual employees change over time and are various. Therefore, even when the same treatment is executed, the same effect is not exhibited. Therefore, to obtain a sufficient treatment effect, it is necessary to provide treatment effect prediction that is suitable for a change in effect factors or state of mind and body of each of treatment target people (users) and a treatment that can be continuously executed.

PTL 1 (US2019/0259500A) describes a technique of detecting a behavior change of a user and providing a treatment based on a rule-based determination.

### SUMMARY OF INVENTION

### Technical Problem

However, with the rule-based determination described in PTL 1, there is a high possibility that a change in effect factors or state of mind and body of each of users cannot be managed, and it is difficult to provide a treatment that can be continuously executed. Therefore, to provide an appropriate treatment effect prediction result and a treatment that can be continuously executed, it is continuously update a treatment prediction model that is learned by machine learning.

To update the prediction model, it is necessary to continuously collect various measurement data used for machine learning from each of users, and it is difficult to implement the continuous information collection from the viewpoints of a burden on the user and the collection cost.

An object of the present invention is to provide a technique of acquiring behavior history data of a user from an electronic device that is used during work or daily life, converting the acquired behavior history data into a feature of measurement data, and learning a prediction model.

### Solution to Problem

A representative example of the present invention disclosed in the present application is as follows. That is, there is provided an information processing system that supports selection of a treatment for a user, the information processing system being configured by a computer including an arithmetic device configured to execute a predetermined process and a storage device connected to the arithmetic device, the storage device storing behavior history data of a user and measurement data of the user, and the information processing system including: a behavior history data feature extraction unit in the arithmetic device configured to extract a behavior history data feature that is a feature of the behavior history data acquired from the user; a measurement data feature extraction unit in the arithmetic device configured to extract a measurement data feature that is a feature of the measurement data acquired from the user; a feature conversion learning unit in the arithmetic device configured to learn a feature conversion model for deriving a feature of measurement data from the behavior history data using the behavior history data feature and the measurement data feature; and a treatment prediction learning unit in the arithmetic device configured to generate a prediction model for providing an appropriate treatment to a user using a first feature extracted from the measurement data, a second feature converted from the behavior history data, the treatment, and an effect of the treatment.

### Advantageous Effects of Invention

According to one aspect of the present invention, an appropriate treatment effect prediction result and a treatment that can be continuously executed can be provided. Objects, configurations, and effects other than those described above will be clarified by describing the following embodiments.

### BRIEF DESCRIPTION OF DRAWINGS

FIG. 1 is a diagram illustrating an example of a hardware configuration of an information processing system according to a first embodiment.

FIG. 2 is a block diagram illustrating a prior learning function that is executed by the information processing system according to the first embodiment.

FIG. 3 is a flowchart illustrating a prior learning process according to the first embodiment.

FIG. 4 is a block diagram illustrating an updated learning function that is executed by the information processing system according to the first embodiment.

FIG. 5 is a flowchart illustrating an updated learning process according to the first embodiment.

FIG. 6 is a block diagram illustrating a treatment prediction function that is executed by the information processing system according to the first embodiment.

FIG. 7 is a flowchart illustrating a treatment prediction process according to the first embodiment.

FIG. 8 is a diagram illustrating an example of measurement items according to the first embodiment.

FIG. 9 is a diagram illustrating an example of measurement items according to the first embodiment.

FIG. 10 is a diagram illustrating an example of a treatment effect presentation result screen according to the first embodiment.

FIG. 11 is a diagram illustrating an example of an activity productivity prediction result screen according to the first embodiment.

FIG. 12 is a diagram illustrating an example of a treatment candidate selection screen according to the first embodiment.

FIG. 13 is a diagram illustrating an example of a treatment determination result screen according to the first embodiment.

### DESCRIPTION OF EMBODIMENTS

Hereinafter, an embodiment of the present invention will be described in detail based on the drawings. In all the diagrams for describing the embodiment, basically, the same functions or processes are represented by the same reference numerals, and the description thereof will not be repeated.

The embodiment of the present invention relates to an information processing method including: a step of extracting a feature of behavior history data acquired from a target person (user) for a treatment; a step of converting the feature of the behavior history data into a feature of plural kinds of measurement data using a machine-learned conversion model; a step of updating a treatment prediction model using the converted feature, the treatment prediction model being previously learned using the plural kinds of measurement data; and a step of outputting a predictive value of a treatment effect of a treatment.

Hereinafter, a specific configuration example of an information processing system that executes the information processing method according to the embodiment of the present invention will be described in detail.

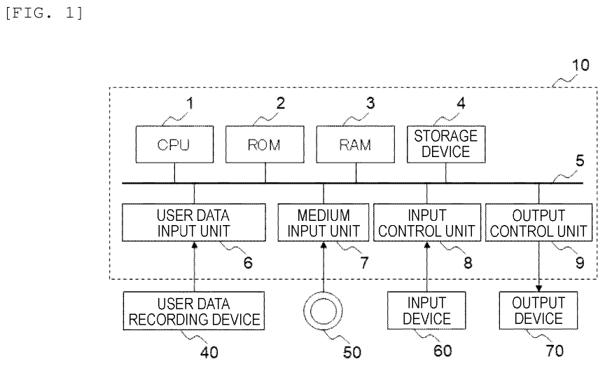

FIG. 1 is a diagram illustrating an example of a hardware configuration of an information processing system according to a first embodiment.

The information processing system according to the embodiment includes a CPU (processor) 1, a ROM (read-only data storage medium configured with a non-volatile memory) 2, a RAM (readable and writable data storage medium configured with a volatile memory) 3, a non-volatile storage device 4, a user data input unit 6, a medium input unit 7, an input control unit 8, and an output control unit 9. Such configurations are connected to each other via a bus 5. An output device 70 is connected to the output control unit 9.

At least one of the ROM 2 or the RAM 3 stores a program, data, and a prediction model required to implement an operation of the information processing system in arithmetic processing of the CPU 1. The CPU 1 executes various processes of the information processing system described below by executing the program stored in at least one of the ROM 2 or the RAM 3. The program that is executed by the CPU 1 may be stored in advance in, for example, a storage medium 50 and may be configured to be read by the medium input unit 7 such as an optical disk drive and stored in the RAM 3. The program may be stored in the storage device 4 and may be loaded from the storage device 4 to the RAM 3. The program may be stored in the ROM 2 in advance.

The user data input unit 6 is an interface for taking in various measurement data of a user recorded in a user data recording device 40. The storage device 4 is a magnetic storage device that stores user data or the like input through the user data input unit 6. The storage device 4 is configured with a non-volatile semiconductor storage medium such as a flash memory or with a magnetic disk drive. The storage device 4 may be an external storage device connected via a network or the like.

The input device 60 is a device that receives an operation of a user, and examples thereof include a keyboard, a trackball, and an operation panel. The input control unit 8 is an interface that receives an operation input input by a user. The operation input received by the input control unit 8 is processed by the CPU 1. The output control unit 9 outputs, for example, the result of the arithmetic processing by the CPU 1 (for example, a prediction result of a treatment recommended for a user and a treatment effect) to the output device 70.

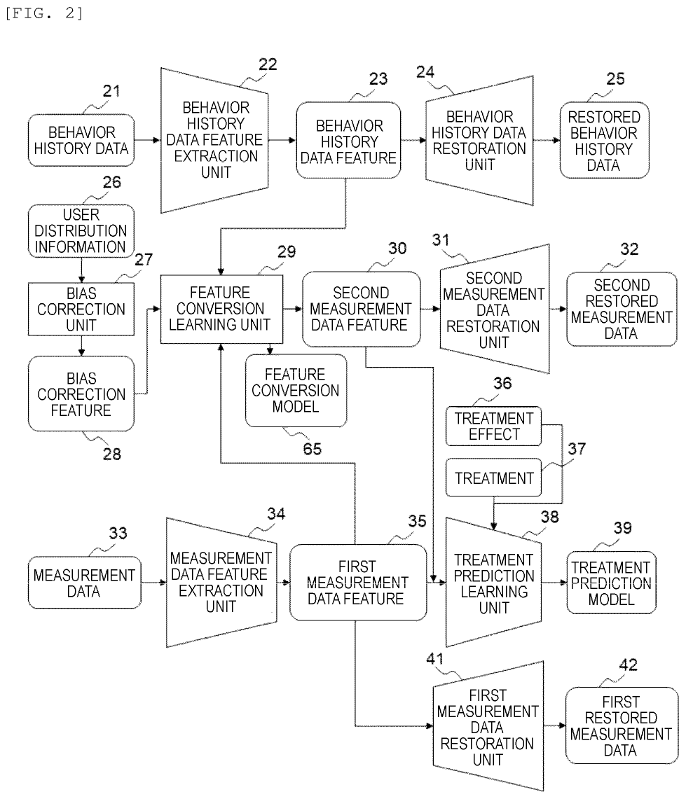

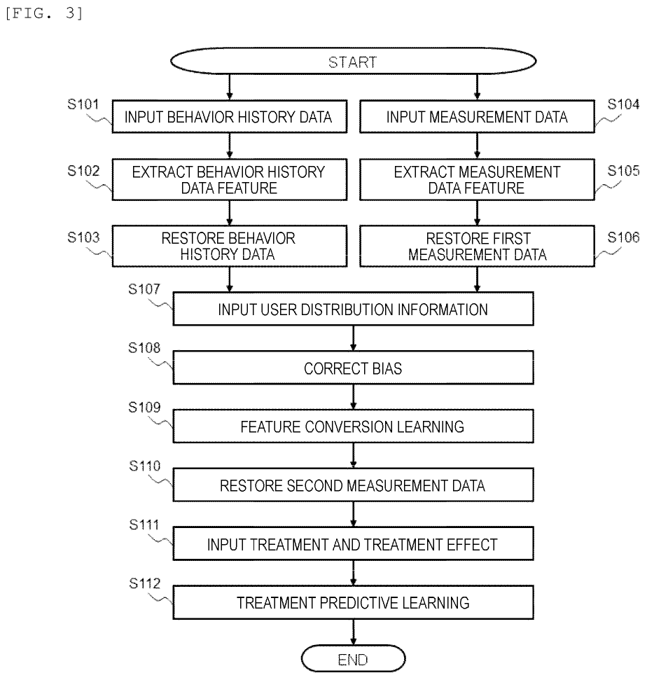

FIG. 2 is a block diagram illustrating a prior learning function of generating a model used in a feature conversion function and a prediction function that are executed by the information processing system according to the embodiment, and FIG. 3 is a flowchart illustrating a process of previously learning a feature conversion model and a treatment prediction model. Next, an operation process of the learning function and the feature conversion function will be described with reference to FIGS. 2 and 3.

First, in Step S101, a behavior history data feature extraction unit 22 receives behavior history data 21 of a user. The behavior history data 21 is an operation log of a device for operation, an operation log of an electronic device for daily life, or a behavior history of a user recorded by an electronic device, and examples thereof include an operation log of a machine in a factory, a driving operation log of a vehicle, an operation log of a personal computer or a smartphone that is simply measurable data in a user's life, and behavior data (for example, acceleration data) recorded in a wearable terminal.

In Step S102, the behavior history data feature extraction unit 22 extracts a behavior history data feature 23 using an encoder function in an autoencoder method of machine learning. In Step S103, a behavior history data restoration unit 24 restores the behavior history data 21 to generate restored behavior history data 25 using a decoder function in the autoencoder method. A method of the feature extraction and the data restoration using the autoencoder will be described below. Whether an appropriate feature is extracted can be verified by comparing the restored behavior history data 25 and the original behavior history data 21 to each other. As such, Step S103 is an option, and when the verification is unnecessary, Step S103 can be skipped.

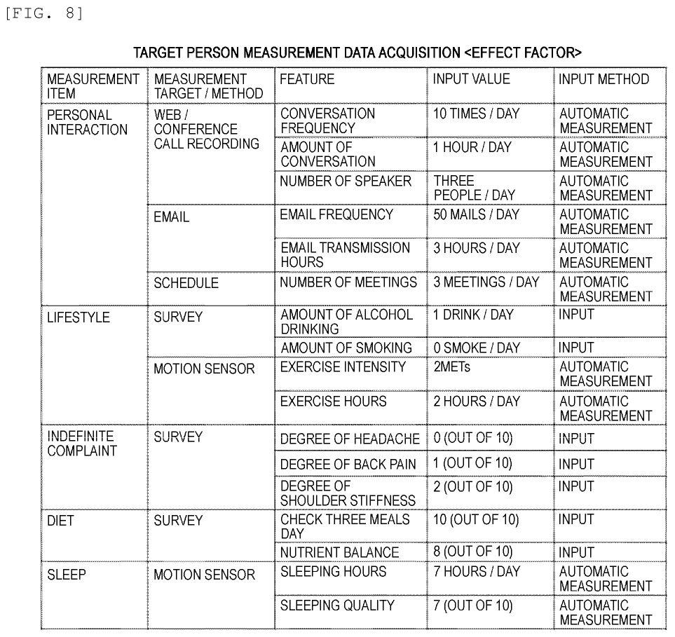

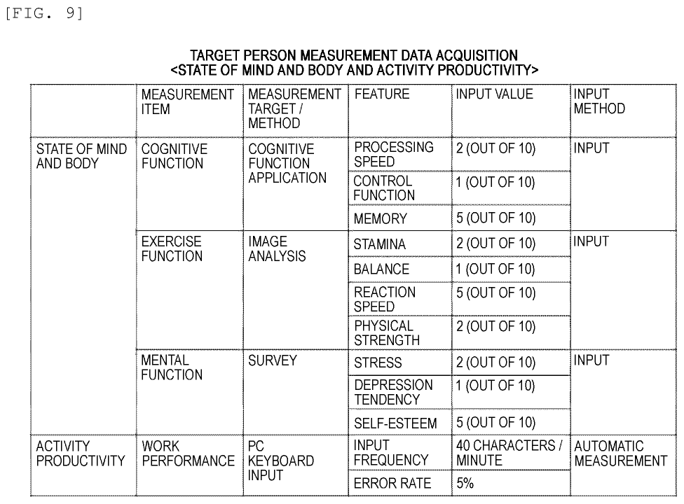

In Step S104, a measurement data feature extraction unit 34 receives measurement data 33 of a user. The measurement data 33 is vital data, exercise function test data, cognitive function test data, or productivity measurement data, and examples thereof include vital data such as blood pressure or heart rate acquired from a wearable device, a medical check-up, a medical institution, or the like, a result obtained in an exercise function test (for example, grip strength, sit-up, standing forward bending, whole body reaction time, one-leg standing with eyes closed, maximum oxygen intake, squat, or balance), a cognitive function test (for example, orientation of time where date and time is answered, clue reproduction where a memory is reproduced, or clock drawing where a clock face is drawn), an answer to a productivity analysis survey, and a keyboard operation pattern during work. More specifically, measurement items illustrated in FIGS. 8 and 9 are measured.

In Step S105, the measurement data feature extraction unit 34 extracts a first measurement data feature 35 using the encoder function in the autoencoder method. In Step S106, a first measurement data restoration unit 41 restores the measurement data 33 to generate first restored measurement data 42 using the decoder function in the autoencoder method. Whether an appropriate feature is extracted can be verified by comparing the first restored measurement data 42 and the original measurement data 33 to each other. As such, Step S106 is an option, and when the verification is unnecessary, Step S106 can be skipped.

Next, in Step S107, a bias correction unit 27 receives user distribution information 26. In Step S108, the bias correction unit 27 generates a bias correction feature 28 to correct a bias of user data. For example, the feature can be corrected by using numerical values shared with the behavior history or the measurement data, for example, a male-to-female ratio, an age distribution, a disease, a smoking habit, and the like among a population of users.

In Step S109, a feature conversion learning unit 29 receives the behavior history data feature 23 and the first measurement data feature 35 and learns to convert the behavior history data feature 23 into the first measurement data feature 35 using the autoencoder method to generate a feature conversion model 65. The feature conversion learning unit 29 receives the bias correction feature 28 and executes the bias correction to generate a second measurement data feature 30 by adding the corrected feature to the behavior history data feature 23.

In Step S110, a second measurement data restoration unit 31 receives the second measurement data feature 30 and learns to restore the measurement data 33 from the second measurement data feature 30 using the decoder. As a result, second restored measurement data 32 is generated.

In Step S111, a treatment prediction learning unit 38 receives a treatment 37 provided to a user and a treatment effect 36 of the treatment policy. The treatment prediction learning unit 38 receives the first measurement data feature 35 and the second measurement data feature 30.

In Step S112, the treatment prediction learning unit 38 predicts a treatment effect of each of treatments and generates a treatment prediction model 39 using the first measurement data feature 35, the second measurement data feature 30, the treatment 37, and the treatment effect 36 such that an appropriate treatment can be provided to the user.

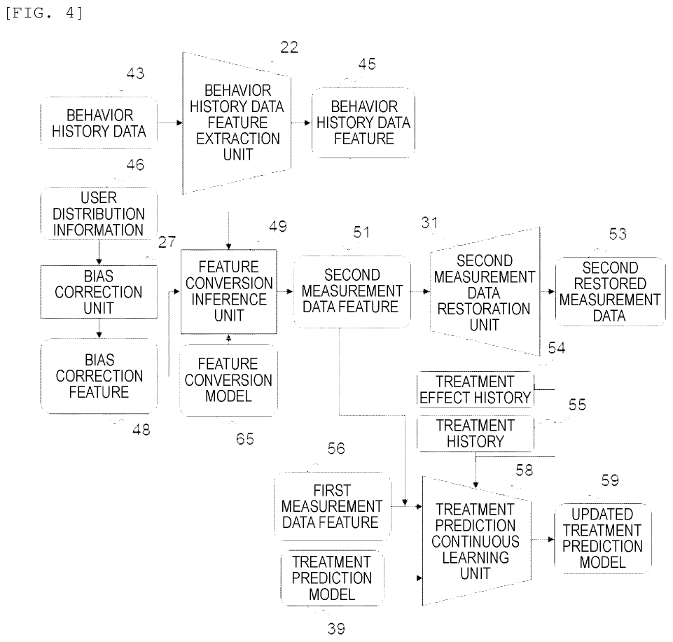

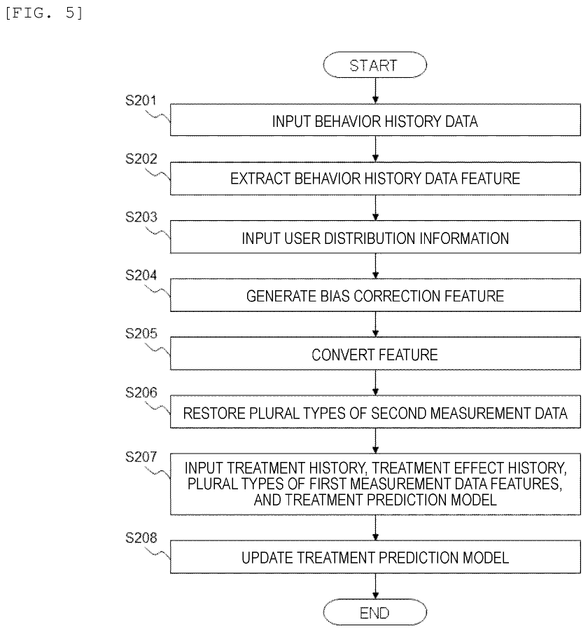

FIG. 4 is a block diagram illustrating an updated learning function where the information processing system according to the embodiment executes a treatment of a user in operation using the treatment prediction model 39, and FIG. 5 is a flowchart illustrating a process of updating and learning the treatment prediction model. Next, an operation process of the updated learning function will be described using FIGS. 4 and 5.

In Step S201, the behavior history data feature extraction unit 22 receives behavior history data 43 of a user. In step S202, the behavior history data feature extraction unit 22 extracts a behavior history data feature 45.

In Step S203, the bias correction unit 27 receives user distribution information 46. In Step S204, the bias correction unit 27 generates a bias correction feature 48 to correct a bias of user data.

In Step S205, a feature conversion inference unit 49 receives the behavior history data feature 45 and executes inference of converting the behavior history data feature into a second measurement data feature 51 using the feature conversion model 65. The feature conversion inference unit 49 receives the bias correction feature 48 and executes the bias correction to generate a second measurement data feature 51 by adding the corrected feature to the second measurement data feature 51.

In Step S206, the second measurement data restoration unit 31 receives the second measurement data feature 51 and generates second restored measurement data 53 from the second measurement data feature 51.

In Step S207, a treatment prediction continuous learning unit 58 receives the second measurement data feature 51, a treatment effect history 54, a treatment history 55, a first measurement data feature 56, and the prior-learned treatment prediction model 39. In Step S208, the treatment prediction continuous learning unit 58 predicts a treatment effect of each of treatments according to a transition state or a treatment history of a user and updates the treatment prediction model 39 to generate an updated treatment prediction model 59 using the first measurement data feature 56, the second measurement data feature 51, the treatment history 55, and the treatment effect history 54 such that an appropriate treatment can be provided to the user.

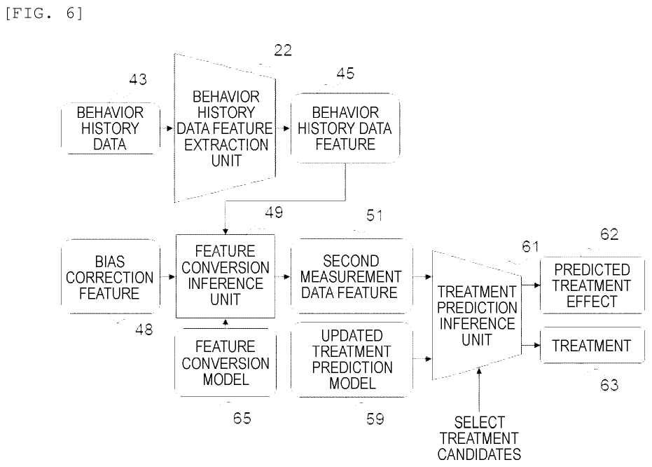

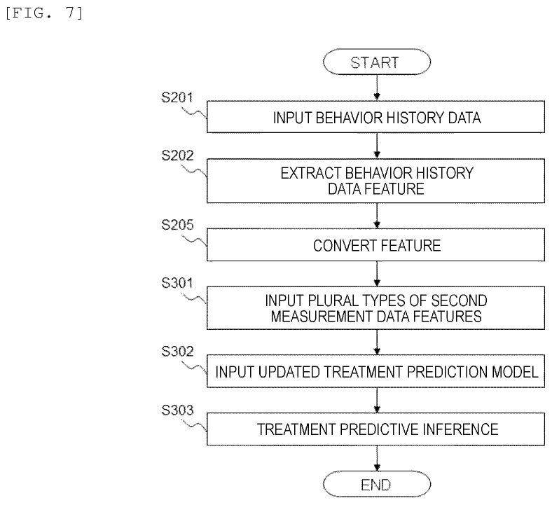

FIG. 6 is a block diagram illustrating a treatment prediction function where the information processing system according to the embodiment executes the treatment prediction using the updated treatment prediction model 59, and FIG. 7 is a flowchart illustrating a process of executing the treatment prediction using the treatment prediction model. Next, an operation process of the treatment predictive inference function will be described with reference to FIGS. 6 and 7.

In Step S201, the behavior history data feature extraction unit 22 receives behavior history data 43 of a user. In step S202, the behavior history data feature extraction unit 22 extracts a behavior history data feature 45.

In Step 205, a feature conversion inference unit 49 receives the behavior history data feature 45 and executes inference of converting the behavior history data feature 45 into the first measurement data feature 56 using the feature conversion model 65. The feature conversion inference unit 49 receives the bias correction feature 48, corrects the bias to the changed feature, and generates the second measurement data feature 51.

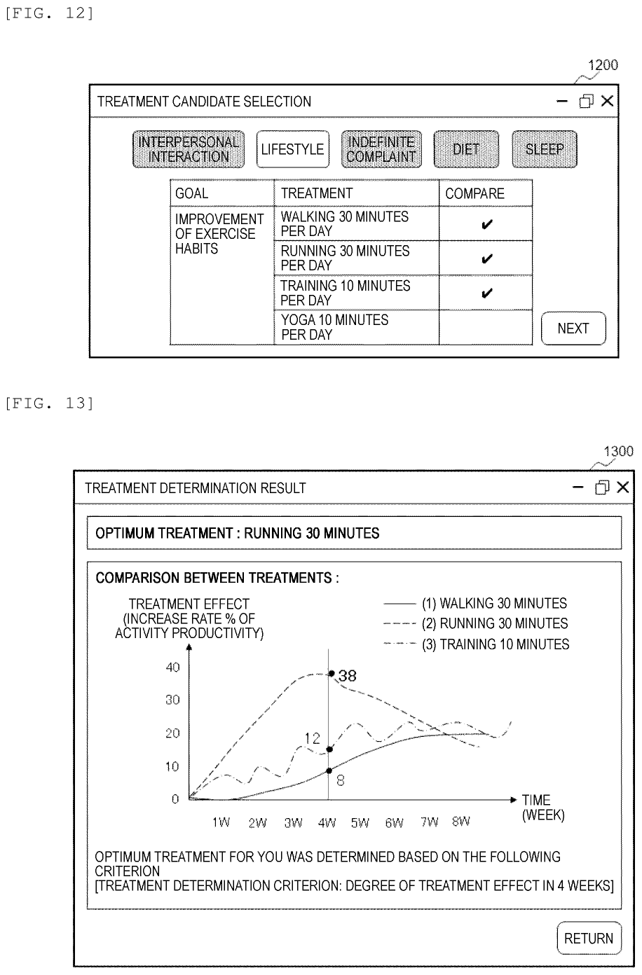

In Steps S301 and S302, a treatment predictive inference unit 61 receives the second measurement data feature 51, the updated treatment prediction model 59, and a selection result (refer to FIG. 12) of treatment candidates. In Step S303, the treatment predictive inference unit 61 outputs a treatment 63 that is provided to the user and a predictive treatment effect 62 that is a treatment effect of the treatment.

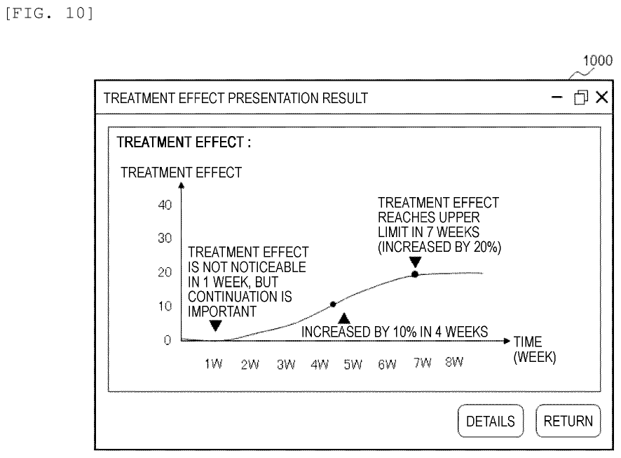

FIG. 10 is a diagram illustrating an example of a treatment effect presentation result screen 1000 that is output from the information processing system according to the embodiment.

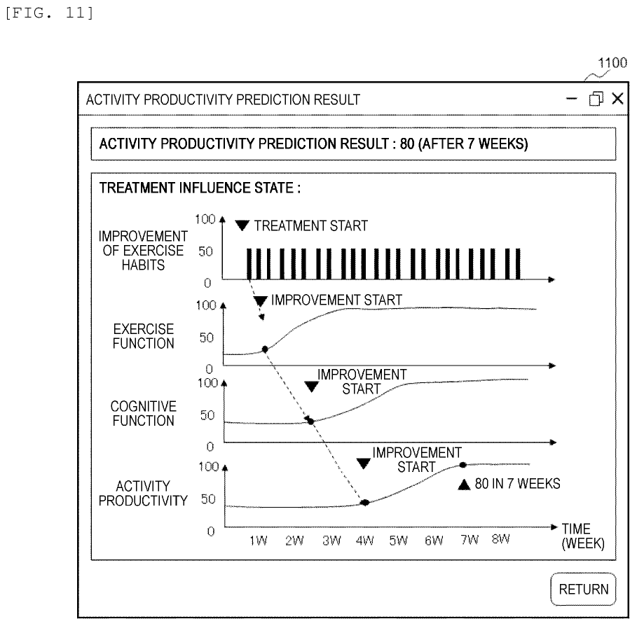

The treatment effect presentation result screen 1000 shows a time-series comprehensive treatment effect (for example, an increase rate of activity productivity expressed in percentage) together with messages at main points. Specifically, depending on a change of the treatment effect, “treatment effect is not noticeable in 1 week, but continuation is important” is shown at the time point of 1 week, “increased by 10% in 4 weeks” is shown at the time point of 4 weeks, and “treatment effect reaches the upper limit in 7 weeks (increased by 20%)” is shown at the time point of 7 weeks. By seeing the treatment effect presentation result screen 1000, the user can recognize the effect of the treatment and can maintain motivation to continue the treatment. A message to be displayed may change depending on the state of the user. By operating a “Detail” button on the treatment effect presentation result screen 1000, an activity productivity prediction result screen 1100 (FIG. 11) is displayed such that the detailed effect of the treatment can be seen.

FIG. 11 is a diagram illustrating an example of the activity productivity prediction result screen 1100 that is output from the information processing system according to the embodiment.

The activity productivity prediction result screen 1100 shows the summary of the treatment effect of the treatment in the upper portion. Specifically, the activity productivity prediction result screen 1100 shows that, although the activity productivity is 50 or less at the start of the treatment, the activity productivity is improved to 80 after 7 weeks and the treatment effect of improving the measurement data is shown.

In the lower portion of the activity productivity prediction result screen 1100, the details of the treatment effect, that is, the improvement of the measurement data by the treatment are shown. Specifically, due to the improvement of exercise habits, the exercise function starts to improve after almost 1 week from the start of the treatment, the cognitive function starts to improve after 2 weeks, the activity productivity starts to improve after 4 weeks, and the activity productivity is improved to 80 after 7 weeks.

FIG. 12 is a diagram illustrating an example of a treatment candidate selection screen 1200 that is output from the information processing system according to the embodiment.

In the upper portion of the treatment candidate selection screen 1200, classifications (interpersonal interaction, lifestyle, indefinite complaint, diet, or sleep) of the treatment candidates are shown, and the user selects a classification of treatment candidates from the classifications. The drawing illustrates a state where “lifestyle” is selected. In the lower portion of the treatment candidate selection screen 1200, specific treatments in the selected classification are presented, and by the user selecting the treatments in the comparison field, the treatment effects can be compared and displayed on a treatment determination result screen 1300 illustrated in FIG. 13.

FIG. 13 is a diagram illustrating an example of the treatment determination result screen 1300 that is output from the information processing system according to the embodiment.

The treatment determination result screen 1300 shows an optimum treatment having the highest effect in the upper portion. In the lower portion of the treatment determination result screen 1300, a difference between the treatment effects of the treatment candidates (activity productivity increase rates expressed in percentage) is shown. Specifically, the drawing shows that, after 4 weeks from the start of the treatment, the treatment effect is improved by 8% by (1) walking 30 minutes, is improved by 38% by (2) running 30 minutes, and is improved by 12% by (3) training 10 minutes.

In the information processing system according to the embodiment of the present invention, for example, a middle-aged employee of a company is set as a target, at least one (desirably a combination of two or more) among at least vital data, exercise function test data, cognitive function test data, and productivity measurement data (survey response record) is collected in advance as plural kinds of measurement data 33, and at least one of an operation log of an electronic device for operation, an operation log of an electronic device for daily life, or a behavior history of a user recorded by an electronic device is collected as the behavior history data 21, and the treatment prediction model 39 is learned. In the information processing system according to the embodiment, to improve the productivity of the employee, while reducing a burden on the employee, the behavior history data 21 that can be easily measured is collected, the collected behavior history data 21 is converted into features of plural kinds of measurement data using the feature conversion model 65 with high accuracy, and an effect of a treatment is predicted using the treatment prediction model 39. Depending on a behavior change transition state, a state of mind and body, a productivity state, a treatment history, and the like of the employee as the target of the treatment, the treatment prediction model 39 is continuously updated, and an appropriate treatment can be provided.

As described above, the information processing system according to the embodiment includes: the behavior history data feature extraction unit 22 configured to extract the behavior history data feature 23 that is a feature of the behavior history data 21 acquired from the user; the measurement data feature extraction unit 34 configured to extract the first measurement data feature 35 that is a feature of the measurement data 33 acquired from the user; the feature conversion learning unit 29 configured to learn the feature conversion model 65 using the behavior history data feature 23 and the first measurement data feature 35 to derive the second measurement data feature 30 from the behavior history data 21; and the treatment prediction learning unit 38 configured to generate the treatment prediction model 39 for providing an appropriate treatment to a user using the first measurement data feature 35 extracted from the measurement data 33, the second measurement data feature 30 converted from the behavior history data 21, the treatment 37, and the effect 36 of the treatment. Therefore, even in the process of the treatment, the prediction model can be updated appropriately along with the elapse of time, and an appropriate treatment effect prediction result and a treatment that can be continuously executed can be provided. A treatment prediction model can be learned using behavior history data of a user from an electronic device that is used during work or daily life.

The information processing system according to the embodiment further includes: the feature conversion inference unit 49 configured to convert the behavior history data feature 23 into the second measurement data feature 30 using the feature conversion model 65; and the treatment prediction continuous learning unit 58 configured to update the treatment prediction model 39 using the converted second measurement data feature 30, the treatment history 55, and the treatment effect history 54 to generate the updated treatment prediction model 59. Depending on a transition state or a treatment history of a user, a treatment prediction model can be learned using behavior history data of the user from an electronic device that is used during work or daily life. The treatment prediction model can be continuously learned using data different from that of prior learning, and the accuracy of the treatment prediction model can be improved while reducing a burden on the user.

The information processing system according to the embodiment further includes: the feature conversion inference unit 49 configured to convert the behavior history data feature 23 into the second measurement data feature 30 using the feature conversion model 65; and the treatment predictive inference unit 61 configured to derive the treatment 63 and the predictive treatment effect 62 using the updated treatment prediction model 59 from the second measurement data feature 51 converted from the behavior history data 43. Therefore, a treatment effect of each of treatments is predicted using behavior history data of a user from an electronic device that is used during work or daily life such that an appropriate treatment can be provided to the user.

The information processing system according to the embodiment further includes: the first measurement data restoration unit 41 configured to restore the measurement data 42 based on the first feature extracted from the measurement data 33; and the second measurement data restoration unit 31 configured to restore the measurement data 32 based on the second measurement data feature 30 extracted from the behavior history data 21. Therefore, whether the feature is appropriately extracted can be verified based on the restored data.

The present invention is not limited to the embodiment and includes various modification examples and identical configurations within the scope of the appended claims. For example, the embodiments have been described in detail in order to easily describe the present invention, and the present invention is not necessarily to include all the configurations described above. Some of the configurations of one embodiment may be replaced with the configurations of another embodiment. Some of the configurations of one embodiment may be added to the configurations of another embodiment. Addition, deletion, and replacement of another configuration can be made for a part of the configuration each of the embodiments.

Some or all of the above-described respective configurations, functions, processing units, processing means, and the like may be implemented by hardware, for example, by designing an integrated circuit. The respective configurations, functions, and the like may be realized by software by a processor interpreting and executing a program that realizes each of the functions.

Information of a program, a table, a file, or the like that implements each of the functions can be stored in a storage device such as a memory, a hard disk, or a solid state drive (SSD) or a recording medium such as an IC card, an SD card, or a DVD.

The drawings illustrate control lines or information lines as considered necessary for explanations but do not illustrate all control lines or information lines required on the actual production line. It can be considered that almost of all components are actually interconnected.

1. An information processing system that supports selection of a treatment for a user,
the information processing system being configured by a computer including an arithmetic device configured to execute a predetermined process and a storage device connected to the arithmetic device, and
the storage device storing behavior history data of a user and measurement data of the user,
the information processing system comprising:
a behavior history data feature extraction unit in the arithmetic device configured to extract a behavior history data feature that is a feature of the behavior history data acquired from the user;
a measurement data feature extraction unit in the arithmetic device configured to extract a measurement data feature that is a feature of the measurement data acquired from the user;
a feature conversion learning unit in the arithmetic device configured to learn a feature conversion model for deriving a feature of measurement data from the behavior history data using the behavior history data feature and the measurement data feature; and
a treatment prediction learning unit in the arithmetic device configured to generate a prediction model for providing an appropriate treatment to a user using a first feature extracted from the measurement data, a second feature converted from the behavior history data, the treatment, and an effect of the treatment.

2. The information processing system according to claim 1, further comprising:
a feature conversion inference unit in the arithmetic device configured to convert the feature of the behavior history data into a feature of the measurement data using the feature conversion model; and
a treatment prediction continuous learning unit configured to update the prediction model using the converted feature of the measurement data, a history of the treatment, and a history of the effect of the treatment.

3. The information processing system according to claim 1, further comprising:
a feature conversion inference unit in the arithmetic device configured to convert the feature of the behavior history data into a feature of the measurement data using the feature conversion model; and
a treatment predictive inference unit in the arithmetic device configured to derive a treatment and a predictive value of an effect of the treatment using the prediction model from the second feature converted from the behavior history data.

4. The information processing system according to claim 1, wherein
the behavior history data includes at least one of an operation log of an electronic device for operation, an operation log of an electronic device for daily life, or a behavior history of a user recorded by an electronic device.

5. The information processing system according to claim 1, wherein
the measurement data includes at least one of vital data, exercise function test data, cognitive function test data, or productivity measurement data of a user.

6. The information processing system according to claim 1, further comprising:
a first measurement data restoration unit configured to restore measurement data based on the first feature extracted from the measurement data; and
a second measurement data restoration unit configured to restore measurement data based on the second feature extracted from the behavior history data.

7. An information processing method of supporting selection of a treatment for a user using an information processing system,
the information processing system being configured by a computer including an arithmetic device configured to execute a predetermined process and a storage device connected to the arithmetic device, and
the storage device storing behavior history data of a user and measurement data of the user,
the information processing method comprising:
a behavior history data feature extraction procedure in the arithmetic device of extracting a behavior history data feature that is a feature of the behavior history data acquired from the user;
a measurement data feature extraction procedure in the arithmetic device of extracting a measurement data feature that is a feature of the measurement data acquired from the user;
a feature conversion learning procedure in the arithmetic device of learning a feature conversion model for deriving the measurement data from the behavior history data using the behavior history data feature and the measurement data feature; and
a treatment prediction learning procedure in the arithmetic device of generating a prediction model for providing an appropriate treatment to a user using a first feature converted from the measurement data, a second feature converted from the behavior history data, the treatment, and an effect of the treatment.

8. The information processing method according to claim 7, further comprising:
a feature conversion inference procedure in the arithmetic device of converting the feature of the behavior history data into a feature of the measurement data using the feature conversion model; and
a treatment prediction continuous learning procedure of updating the prediction model using the converted feature of the measurement data, a history of the treatment, and the effect of the treatment.

9. The information processing method according to claim 7, further comprising:
a feature conversion inference procedure in the arithmetic device of converting the feature of the behavior history data into a feature of the measurement data using the feature conversion model; and
a treatment predictive inference procedure in the arithmetic device of deriving a treatment and a predictive value of an effect of the treatment using the prediction model from the second feature converted from the behavior history data.

10. The information processing method according to claim 7, wherein
the behavior history data includes at least one of an operation log of an electronic device for operation, an operation log of an electronic device for daily life, or a behavior history of a user recorded by an electronic device.

11. The information processing method according to claim 7, wherein
the measurement data includes at least one of vital data, exercise function test data, cognitive function test data, or productivity measurement data of a user.

12. The information processing method according to claim 7, further comprising:
a first measurement data restoration procedure of restoring measurement data based on the first feature converted from the measurement data; and
a second measurement data restoration procedure of restoring measurement data based on the second feature converted from the behavior history data.
